(19) United States
(12) Patent Application Publication
(10) Pub. No.: US 2005/0234322 A1
(43) Pub. Date: Oct. 20, 2005

(54) Surgical site marking assembly and method of using same

(76) Inventor: Stephen Bruce Lober, Athens, GA (US)

Correspondence Address: NEEDLE & ROSENBERG, P.C., SUITE 1000, 999 PEACHTREE STREET, ATLANTA, GA, 30309-3915, US

(21) Appl. No.: 11/086,766

(22) Filed: Mar. 22, 2005

Related U.S. Application Data
(60) Provisional application No. 60/555,379, filed on Mar. 22, 2004.
(60) Provisional application No. 60/637,977, filed on Dec. 20, 2004.

(51) Int. Cl. A61B 5/05

(52) U.S. Cl. 600/407

(57) ABSTRACT

A surgical site marking assembly, comprising a print member having a top surface and a male protrusion formed thereon, the male protrusion having a distal end forming a print face sized and shaped to define a mirror image of a desired image to be transfer printed onto a surgical patient in a manner indicative of a surgical site location.

### CROSS REFERENCE TO RELATED APPLICATIONS

This application claims priority to U.S. Application No. 60/555,379, which was filed Mar. 22, 2004, and to U.S. Application No. 60/637,977, which was filed Dec. 20, 2004, the entire disclosures of which are hereby incorporated by reference in their entirety.

### FIELD OF THE INVENTION

This invention relates generally to a surgical site marking device that enables the uniform and consistent marking on surgical patients in order to indicate the correct surgical site.

### BACKGROUND OF THE INVENTION

The use of marking pens on surgical patients prior to surgery is common. Often a surgeon will use pens to mark lines or designate areas on a patient's body so as to know the proper place for an incision or other surgical procedure that will be performed during the operation. In such cases, it is extremely important that the incision or other surgical procedure be at the proper location. However, with the use of these marking pens, it is often difficult to provide uniform and legible handwritten markings. For example, in some instances, a surgeon or a nurse may mark a particular location with an “X”. This marking could on the one hand be interpreted to mean “X” marks the correct location for a surgical procedure to be performed. On the other hand, the “X” could be interpreted to mean a location where a surgical procedure should not be performed. Accordingly, the use of such pens and hand written marks may result in potentially ambiguous and unclear markings that may ultimately lead to confusion during the surgical procedure.

Typically, a surgeon will use a felt tip pen to mark lines or designate the correct area for the desired surgical procedure. However, because of the patient's perspiration, natural oils and fluids that are used on the patient's body prior to surgery, such as antiseptic solutions, these lines and markings made by the marking pens have a tendency to spread out or “bleed” after being made on the skin. Additionally, once an incision has been made, blood usually spills on the patient's skin, further blurring the lines. While attempts have been made to form these markings with a fine tip, blood and other fluids cause the ink to spread, thereby obscuring the original markings.

Another problem with the marking pens of the prior art is that they have a tendency to dry out. Some pens dry out in the package and others dry out after a single use. Additionally, the felt tip of the pen may get “gummed up” with the betadine used on the patient. Because of these problems, many surgeons have been known to break open a pen and use the ink reservoir inside the pen to draw the lines. The reservoir, however, is fairly broad and results in a substandard marking.

To overcome these problems, there is a need for a surgical marking device which enables a surgeon to consistently place a professional looking, uniform, and unambiguous marking onto a patient in order to clearly designate the correct location for a desired surgical procedure.

### SUMMARY OF THE INVENTION

Among other aspects that will become apparent to one of ordinary skill in the art, the instant invention provides a surgical site marking device that enables the uniform and consistent marking of an image on to the tissue of a surgical patient in order to designate the desired location of surgical procedure.

In one aspect, the present invention provides an image transfer marking assembly for indicating the proper location of a surgical procedure to be performed on a patient about to undergo surgery. In this aspect, the image transfer marking device comprises a print member having a top surface and a male protrusion formed thereon, the male protrusion having a distal end forming a print face sized and shaped to define a mirror image of a desired image to be transfer printed onto a surgical patient in a manner indicative of a surgical site location. In one aspect, the print face may be preloaded with a desired marking agent. Alternatively, in another aspect the print face component may require selective loading by contacting the print face of the print member with a marking agent reservoir containing a suitable marking agent.

In another aspect, the present invention provides a method for non-permanently marking the proper place for a surgical procedure to be performed on a patient's body with a marking device. The method comprises identifying a surgical patient in need of having a surgical site marked; identifying a surgical site on the identified surgical patient; providing a print member having a top surface and a male protrusion formed thereon, the male protrusion having a distal end forming a print face sized and shaped to define a mirror image of a desired image to be transfer printed onto a surgical patient, wherein the print face is at least partially loaded with a suitable marking agent; and contacting the at least partially loaded print face with the identified surgical site to thereby deposit at least a portion of the loaded marking agent onto the surgical site and to provide the desired transfer printed image formed by the deposited marking agent.

In still another aspect, the present invention provides a surgical site marking kit comprising at least one surgical site marking assembly comprising a print member having a top surface and a male protrusion formed thereon, the male protrusion having a distal end forming a print face sized and shaped to define a mirror image of a desired image to be transfer printed onto a surgical patient in a manner indicative of a surgical site location.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a perspective view of a surgical site marking assembly according to one aspect of the present invention.

FIG. 2 is a perspective view of a print member according to one aspect of the present invention.

FIG. 3 is a cross-sectional side view of the surgical site marking assembly of FIG. 1.

FIG. 4 is an exploded perspective view of the surgical site marking assembly of FIG. 1 and FIG. 3.

FIG. 5 is a top plan view of a surgical marking pen blister pack according to one aspect of the present invention.

FIG. 6 is a cross-sectional view of the surgical marking pen blister pack of FIG. 5.

FIG. 7 is a side view of the surgical marking pen blister pack of FIG. 5.

FIG. 8 is a perspective view of a surgical marking pen according to one aspect of the present invention.

FIG. 9 is a top plan view of a surgical site stencil according to one aspect of the present invention.

FIG. 10 is a top plan view of a temporary surgical site marking tattoo or decal according to one aspect of the present invention.

FIG. 11 is a schematic view of a surgical site marking kit according to one aspect of the present invention.

FIG. 12 is a top plan view of a storage subassembly according to one aspect of the present invention.

FIG. 13 is a cross-sectional view of the storage subassembly of FIG. 12.

### DETAILED DESCRIPTION OF THE INVENTION

The present disclosure may be understood more readily by reference to the following detailed description of various aspects of the disclosure and the Examples included therein and to the Figures and their previous and following description.

Before the present articles, devices and/or methods are disclosed and described, it is to be understood that this disclosure is not limited to the specific aspects or embodiments described herein, as such may, of course, vary. It is also to be understood that the terminology used herein is for the purpose of describing particular embodiments only and is not intended to be limiting.

It must be noted that, as used in the specification and the appended claims, the singular forms “a,” “an” and “the” include plural referents unless the context clearly dictates otherwise.

Ranges may be expressed herein as from “about” or “approximately” one particular value, and/or to “about” or “approximately” another particular value. When such a range is expressed, another embodiment includes from the one particular value and/or to the other particular value. Similarly, when values are expressed as approximations, by use of the antecedent “about” or “approximately” it will be understood that the particular value forms another embodiment.

As used herein, the term “optional” or “optionally” means that the subsequently described event or circumstance may or may not occur, and that the description includes instances where said event or circumstance occurs and instances where it does not.

As used herein, the term or phrase “surgical site” may, in one aspect, refer to, without limitation, the exact location where a surgical procedure is to be performed on a surgical patient. Alternatively, the term surgical site may, without limitation, refer to a predetermined location on a surgical patient that is sufficiently near to the exact location of a surgical procedure to be performed. Thus, for example, one of ordinary skill in the art will appreciate that it may not be practical to mark directly on the eye of a patient about to undergo eye surgery. Thus, in this example, a marking assembly or other marking device according the present invention could be used to mark a location near the patient's eye, such as on the desired side of the patient's forehead or above the corresponding eyebrow, in a manner that is still indicative of the desired surgical site.

As set forth above, in one aspect, the present invention provides a surgical site marking assembly 10 comprised of a print member 11 having a top surface 12 and a male protrusion 14 formed thereon. The male protrusion comprises a distal end 16 forming a print face 18 sized and shaped to define a mirror image 17 of a desired image to be transfer printed onto a surgical patient in a manner indicative of a surgical site location. FIG. 2 illustrates an exemplary print member according to the instant invention. The exemplified print face 18 is shaped with the mirror image 17 of the word YES circumferentially surrounded by a border.

As further illustrated in FIG. 2, the print member 11 further comprises a print member body 20, wherein the top surface 12 is formed on a portion of the print member body 20. The print member body, as exemplified, forms a block and is substantially, square in shape. However, one will appreciate that other dimensions and shapes of the print member body portion, such as, for example, circular, oval and rectangular are contemplated. To this end, the particular size and shape of the print member body may be customized to any desired specifcation.

The image to be transfer printed onto the patient's skin may be any desired graphic, alpha, numeric and/or alpha-numeric combination that is suitable for use in a manner that would clearly indicate the correct site of the surgical procedure. In one aspect, it is further envisioned that the desired image may be reflective of the actual surgical procedure to be performed. To this end, it should also be appreciated that any alpha, numeric, or alpha-numeric image, phrase or term may further be configured in any desired font, including without limitation, such fonts as Times Roman, Courier, Arial and the like as well as in conjunction with any one or more special effect such as bold face print, all capital letters, italics, underlined text, and the like. It should also be understood that the print face may in one aspect form the desired image to be transfer printed onto the surgical patient. Alternatively, the print face may define a female depression that forms the mirror image of the desired image. In this aspect, as one will appreciate, the area of the print face surrounding the female depression is the raised surface that imprints the patient's skin. According to this aspect, the marking agent that is deposited onto a surgical patient would appear to surround the desired image, whereas the desired image or mark would appear as unmarked or unprinted natural skin color.

In one aspect, the print face is preferably raised from the top surface of the print member a distance in the range of from between approximately 1 mm to approximately 5 mm. In a more preferred aspect, the print face is raised from the backing a distance in the range of from between approximately 2 mm to approximately 3 mm.

An exemplary image to be transfer printed may, for example, comprise the word “YES.” Thus, as depicted in FIG. 2, the print face 18 may be shaped with the mirror image 17 of the word “YES” such that the desired image to be transfer printed onto a patient's skin comprises the word “YES.” As further depicted in FIG. 2, the print face 18 may be further shaped to provide a border 19 circumferentially surrounding the mirror image of the word “YES” such that the desired image to be transfer printed onto a surgical patient comprises the word “YES” circumferentially surrounded by a border. To this end, it will be appreciated that the desired mark or image to be transfer printed onto a patient's skin will be configured on the marking device in a mirror image arrangement in order to enable the transferred image to appear correctly.

It should also be understood that the phrase or term to be image transferred onto a patient is not limited to a phrase or term of the English language. For example, and without limitation, the image to be transferred may comprise a phrase or term from the Spanish, French, German, Russian, Chinese, Japanese, and/or Arabic languages. In still another aspect, it is also envisioned that the image to be transferred may comprise a phrase or term from two or more languages.

One of skill in the art will further appreciate that the surgical site marking assembly 10 may be scaled up or down to provide any desired size marking. In one aspect, the print face formed on the distal end of the male protrusion may extend for approximately 28 mm in a widthwise dimension. In an alternative aspect, the male protrusion and print face formed on the distal end thereof may be approximately 14 mm wide. Likewise, the print member body may be scaled to any desired shape and size. For example, in one aspect, the print member body has a block-like shape having a length and width of approximately 48 mm and a thickness, or height, of approximately 8 mm.

The print face 18 of the print member may, in one aspect, be pre-loaded with a suitable marking agent 24. The preloading may comprise impregnating or loading the print face with a desired marking agent. However, it should also be appreciated that the impregnation of marking agent into the print face may be achieved, for example, by contacting the print face 18 with a marking agent reservoir 22 comprising a suitable marking agent. Moreover, in still another aspect, it is contemplated that the print member may be prepackaged and stored such that the print face 18 is in contact with a marking agent reservoir 22 in order to provide a preloaded print face.

The print member 11, including the print member body 20 and the male protrusion 14 may be constructed of any known material suitable for use in conventional image transfer devices, such as latex rubber and/or a high density polyurethane foam. However, in a preferred aspect, the print member is comprised of a high density polyurethane foam. An exemplary polyurethane foam suitable for use in the instant invention is the high density foam commercially available from the Heubach Corporation in Garland, Tex. The use of polyurethane foam construction may provide the added benefit to those surgical patients who have allergic reactions to latex and other rubber materials commonly utilized in the medical profession. It is further contemplated by the instant invention that the print member, including the print member body and the male protrusion forming the print face, may be constructed of materials that minimize undesired wicking of a marking agent through the male protrusion and into the member body portion. For example, in one aspect, the male protrusion 14 is comprised of a high density polyurethane foam which is substantially impervious to excess absorption of marking agent. To this end, in one aspect of the invention, the male protrusion may be constructed of a material capable of preventing wicking of marking agent into the print member body. Alternatively, it is further contemplated that the print member body 20 may be comprised of a material that is at least substantially impervious to a marking agent. Furthermore, in still another aspect, it is contemplated that a coating or layer that is impervious to the marking agent may be deposited on the top surface of the print member and positioned intermediate to the print member body and the male protrusion. Thus, in accordance with this aspect, the impervious coating layer may block and prevent undesired wicking of the marking agent through the top surface of the print member and into the print member body portion.

Suitable marking agents 24 are preferably those approved for use on a patient's skin and those that are temporary (i.e. biodegradable) such that the patient is not left with a prolonged tattoo marking the desired location for the surgical procedure. To this end, suitable marking agents may include inks that have been approved by the United States Food and Drug Administration (FDA), including without limitation, inks that have been approved for use with food. In one aspect, the marking agent 24 is an ink composition such as methylene blue, brilliant green and/or gentian violet. Furthermore, it should be understood that any desired color may be used provided that the image, when transferred to the patient's skin, provides a sufficient marking that is clearly visible to the surgeon.

The marking agent reservoir 22 may be any conventional reservoir commonly used in the art to store a suitable marking agent 24. For example, as depicted in FIG. 3, a suitable marking agent reservoir may comprise a conventional foam pad 26 impregnated with a desired marking agent 24. The foam pad may be housed in a storage container 28 fitted with a closure lid 29 to prevent the marking agent from drying out when the foam pad is not in use. If desired, the closure lid may form a hermetic seal 30 with the peripheral side edge 32 which may add further protection from external elements to the impregnated foam pad 26.

In still another aspect, the storage container 28, as depicted in FIG. 12 and FIG. 13, can further comprise a plurality of shoulder members 25 formed in the bottom of the tray recess 34. As depicted, the shoulder members have a height dimension 23 relative to the recess surface 36 that is greater than the height dimension of the marking agent reservoir. Thus, in accordance with this aspect, when a plurality of shoulder members 25 substantially circumferentially surround the ink reservoir 22, the shoulder members function as a guard member restricting at least portions of the top surface of a print member 11 from contacting the marking agent reservoir.

It is should be understood that while the marking agent reservoir may be configured in any desired shape and size, it may, in one aspect, be configured such that the reservoir may adequately receive the print face of the print member to thereby load the print face component with the marking agent.

Additionally, it is contemplated by the instant invention that the container may further comprise a guard member 40 for preventing the undesired loading of ink onto a surface other than the print face of the print member, such as on to the top surface of the print member. To this end, one of skill in the art will appreciate that the presence of ink on surfaces other than the print face component may result in ink being transferred on to a user's hands and/or unwanted smudges and uneven or illegible printing onto a patient's body.

Any conventional means for restricting the top surface of the print member from contacting the impregnated ink pad may be used with the present invention. For example, in one aspect, the storage container 28 housing an ink impregnated pad (forming the reservoir) may further comprise a guard member 40 constructed and arranged to restrict the loading of ink to only the print face of the print member. In this aspect, the guard member may be a restrictor plate 42 that is positioned in overlying registration with the marking agent reservoir. The plate may define an opening 44 that is dimensionally sized and shaped such that the print face of the print member component may “pass” through the opening. Alternatively, the impregnated foam pad 26 may be constructed and arranged such that thickness is less than the thickness of the male protrusion 14 forming the print face on the distal end thereof. In accordance with this aspect, the maximum displacement of the foam pad, when depressed, is less than the depth of displacement required for the top surface of the print member to contact the foam pad.

It should be understood that a surgical site marking assembly 10 according to the instant invention may comprise a separate and independent image transfer print member 11 and a separate and independent marking agent reservoir 22. In accordance with this aspect, a user would first manually load the print face of the print member by contacting the print face with a marking agent reservoir loaded with a suitable marking agent. Alternatively, a marking assembly 10 according to the present invention may comprise a print member 11 that is prepackaged in contact with a suitable marking agent reservoir 22 already containing a marking agent 24. In accordance with this aspect, the print face would be pre-loaded with a suitable marking agent and ready for use when removed from the packaging.

An exemplary and non-limiting surgical site marking assembly according to the present invention is depicted in FIG. 4. As illustrated, a surgical marking assembly 10 may comprise a print member 11 having a top surface and a male protrusion formed thereon, the male protrusion having a distal end forming a print face sized and shaped to define a mirror image of a desired image to be transfer printed onto a surgical patient in a manner indicative of a surgical site location. A storage subassembly comprised of a closure lid 29, a storage tray 28 and a marking agent reservoir 22 may also be provided. The exemplary storage tray comprises a peripheral edge surface 32 adapted to releasably receive a portion of the closure lid and further defines a recess 34 sized and shaped to releasably receive the print member in overlying registration with a recess surface 36 formed therein. The tray may be comprised any conventional polymeric or plastic material conventionally use in connection with medical device packaging.

The marking agent reservoir 22, such as for example the foam pad 26, may be deposited on a portion of the recess surface of the tray and may be loaded with a suitable marking agent. Thus, when the print member is received within the tray recess, as depicted in FIG. 3, the print face formed by the distal end of the male protrusion is also positioned in overlying registration with the marking agent reservoir. Further, the print member 11 also comprises a print member body 20, wherein the top surface is formed on a portion of the print member body 20. Thus, when the print member 11 is received within the tray recess, the print member is releasably restrained by a friction fit formed between at least a portion of the print member body and at least a portion of the tray recess.

The exemplified guard member 40 comprises a restrictor plate 42 defining an opening 44 sized and shaped to receive the male protrusion of the print member. When positioned on at least a portion of the marking agent reservoir, the opening 44 is dimensionally sized to allow the passage of the print face component of the print member such that the print face may contact the marking agent reservoir to load the print face with a suitable marking agent. In contrast, the plate portion of the guard member restricts at least a portion of the top surface of the print member from contacting the marking agent reservoir.

In one aspect, the storage subassembly, including the tray and marking agent reservoir, may be constructed and arranged such that when the print member is received within the tray recess, the print face may be in fluid communication with at least a portion of the marking agent loaded within the marking agent reservoir. Thus, when the closure lid is at least partially removed or opened and the print member is removed from the storage tray for use, the print face may be preloaded with a desired marking agent and immediately ready for use.

As depicted, the storage subassembly may further comprise a closure lid 29 to, at least in part, prevent the marking agent from drying out when the image transfer device is not in use and to prevent dust, dirt and other contaminants from contacting the various components of the assembly prior to use. In one aspect, the lid may be releasably affixed to at least a portion of the peripheral edge surface 32 of the storage subassembly by a hermetic seal 30. An exemplary hermetically sealed closure lid may be comprised of any conventional peelable packing material that is functional in a wide variety of temperatures and that is heat sealable to itself and/or to a wide range of polar and/or non-polar plastics, such as those conventionally used in connection with medical devices and related packaging. In one aspect a preferred closure lid is comprised of the TPC-0815 high barrier, heat sealable, peelable packaging material, commercially available from Tolas Health Care Packaging, in Feasterville, Pa.

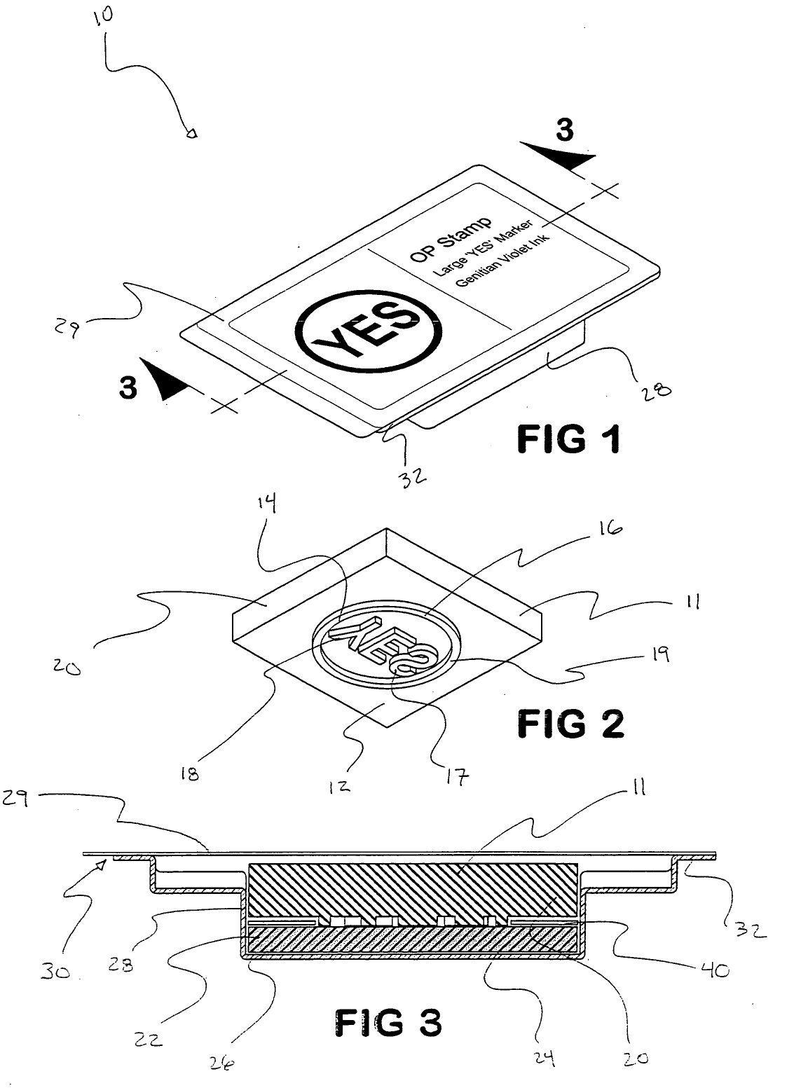

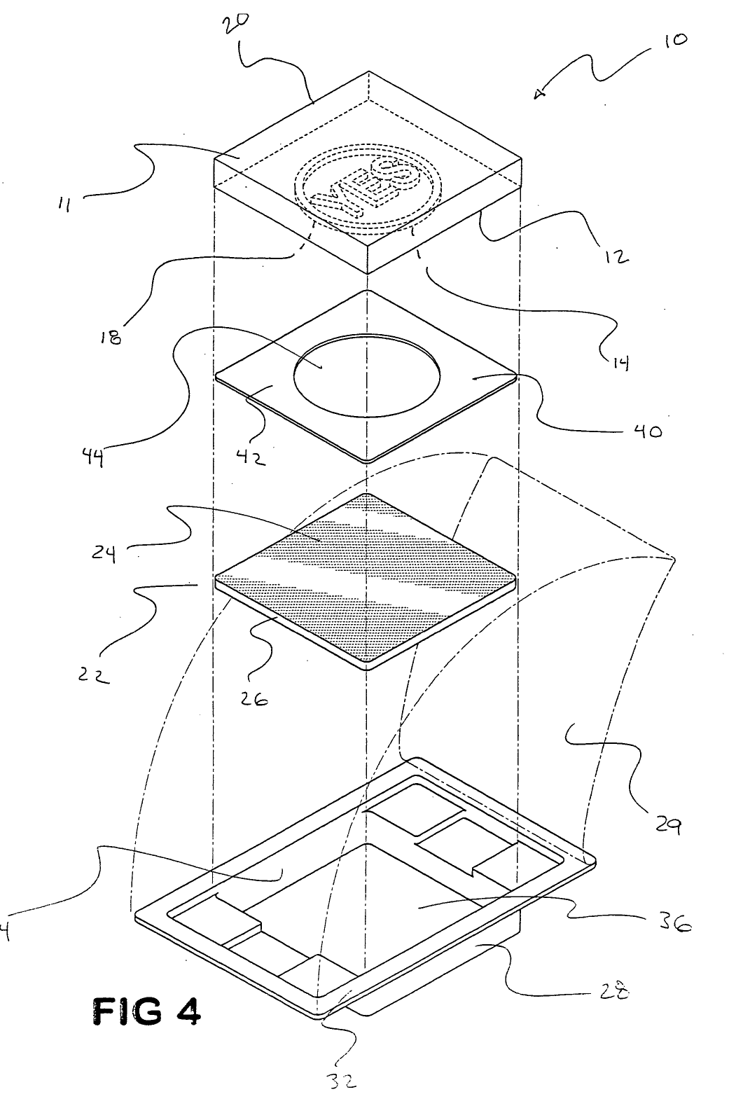

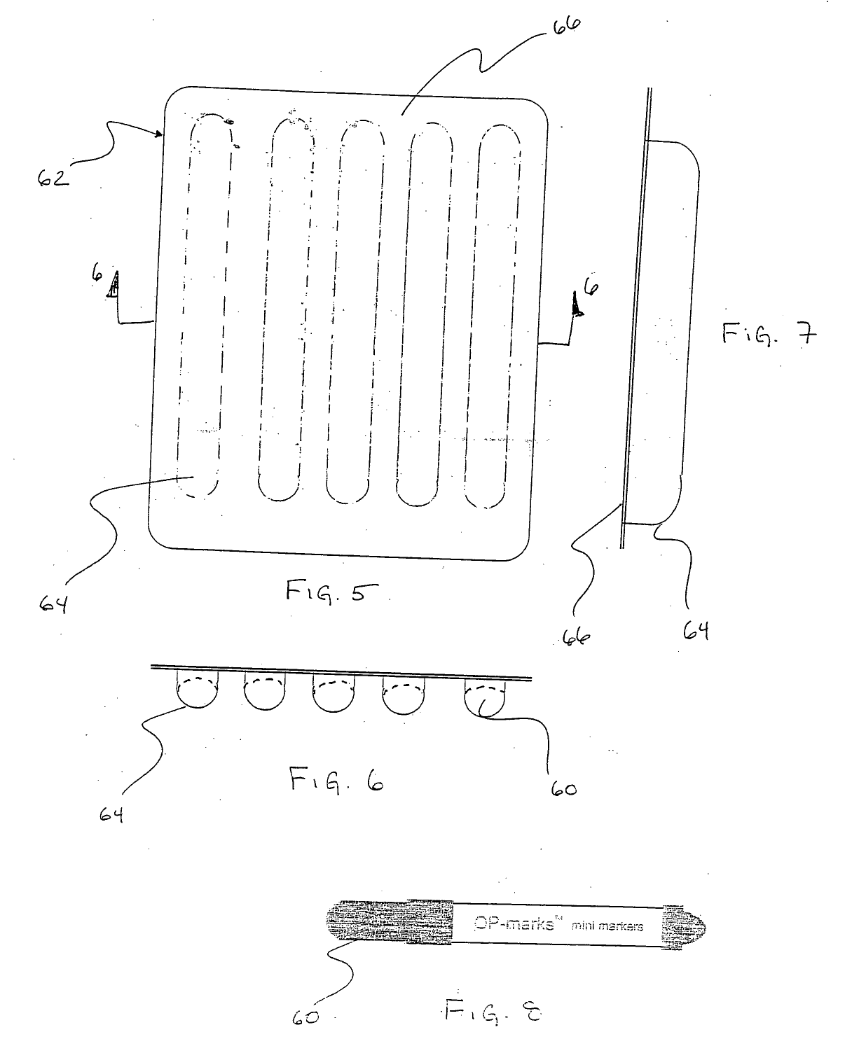

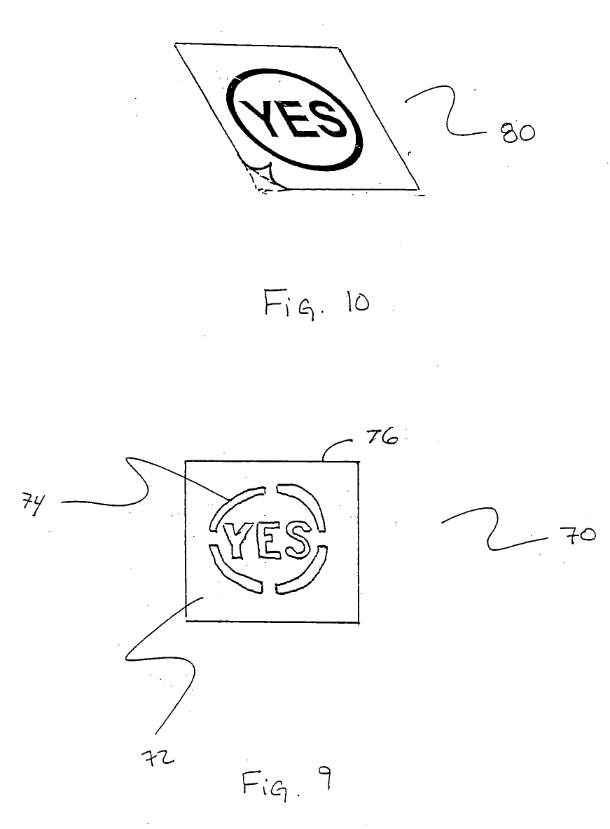

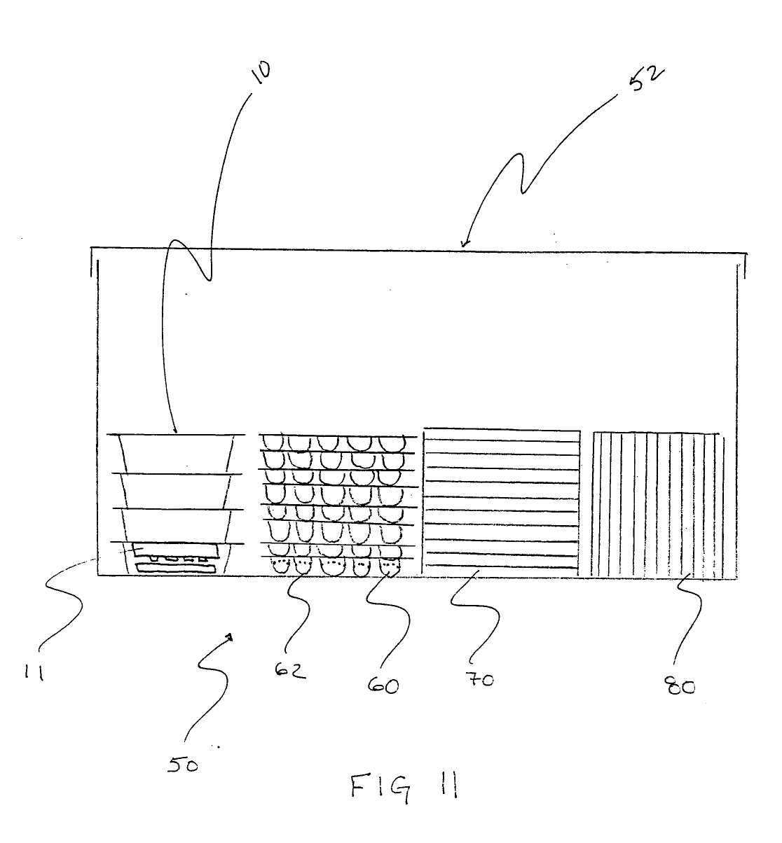

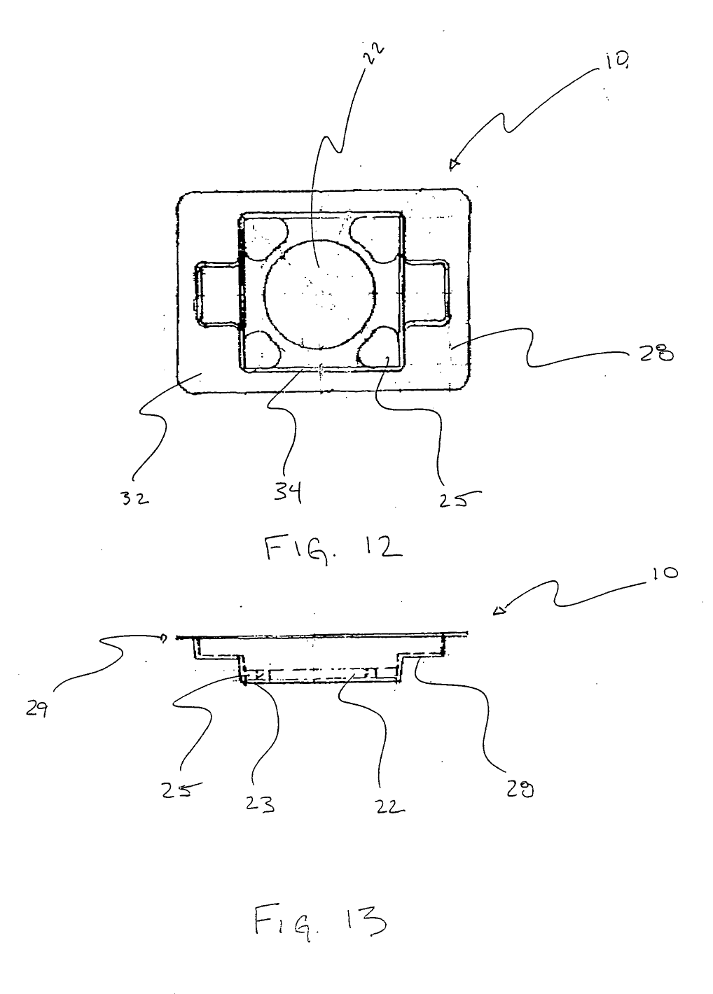

In use, a sealed closure lid 29, as exemplified in FIG. 1., would be at least partially opened by peeling or otherwise tearing the lid away from the peripheral edge 32 of the storage tray 28. Once opened, the print member may then be removed from the storage tray. The print face of the print member being preloaded with a suitable marking agent, may then be positioned in contact with a patient's body with sufficient pressure to deposit marking agent thereon. It is further contemplated that after use, the entire assembly, including print member, storage tray and closure lid, may be then be discarded or disposed of in a conventional manner after a single use. Thus, it will be appreciated up practicing the instant invention that the opening of the closure lid may, in one aspect, provide a signal to a user that a marking assembly has previously been used and should be disposed of. Such evidence of possible previous use may reduce the likelihood that an assembly will be used on a second patient and may thus help to prevent the spread of infectious diseases and other patient to patient contaminations, such as, for example, methycillin-resistant staphylococcus aureus. In one aspect, it is further contemplated that the marking agent reservoir 22 may be loaded with a limited and predetermined amount of a suitable marking agent 24 such that the print face may only be loaded and operable for a predetermined number of uses. For example, and without limitation, a marking agent reservoir may contain enough marking agent to permit between approximately 1 and 20 applications, more particularly between approximately 1 and 10 applications, and still more particularly between approximately 1 and 5 applications.

It is also contemplated that the marking agent reservoir may be loaded with a predetermined amount of marking agent such that once the reservoir is exposed for a predetermined amount of time after the removal of the closure lid, the marking agent will evaporate and/or otherwise dry up and become inoperable in order to ensure that the marking assembly is not used on multiple patients and is instead disposed of after its initial use. However, it should also be understood that an assembly according to the instant invention may, if desired, also be constructed and arrange for multiple uses.

In another aspect, the present invention further provides a surgical site marking kit 50 comprising at least one image transfer print member 11. As depicted in FIG. 11, a kit according to this aspect may further comprise at least one additional conventional surgical site marking device. For example, and without limitation, a kit according to the present invention may comprise at least one surgical site marking pen 60, at least one surgical site marking stencil 70, and/or at least one temporary surgical site marking tattoo or decal 80. Additionally, a kit may optionally contain a sealable packaging enclosure 52 adapted to receive one or more component parts of the kit, a marking agent reservoir, instructions for use and/or additional marketing materials or literature. To this end, instructions optionally included within a kit of the instant invention would, in one aspect, be in substantial accordance with conventional marking surgical marking protocols known to one of ordinary skill in the art. Such marking procedures may include those procedures and protocols recommended by the Association of Preoperative Registered Nurses and/or the Joint Commission on Accreditation for Health Care Organizations.

A surgical site marking pen 60 suitable for use in a kit of the instant invention may be any conventional marking pen that may be held freely in a user's hand, similar to a conventional writing instrument, and which is suitable for use in marking a surgical site on a surgical patient. Thus, it is contemplated that a surgical site marking pen according to the instant invention may be used for drawing lines, words, symbols and/or any other desired mark on both soft and hard tissue of a surgical patient. An exemplary and commercially available marking pen suitable for use in the instant invention is manufactured by and commercially available from Chief Ling Enterprise Co., Ltd., Chang Hua Hsien, Taiwan 515 R.O.C.

In an exemplary aspect, and without limitation, a conventional surgical site marking pen 60 according to the instant invention comprises an elongated housing having a hollow interior portion and two ends, one end of which is closed and the opposite end has an opening which may receive a conventional nib. The elongated housing may be cylindrical or tubular in shape. Alternatively, the elongated housing could be angular such as triangular in shape, which one of ordinary skill in the art will appreciate may inhibit the rolling of the marker. The interior of the elongated housing further comprises a suitable marking agent reservoir. The nib may be fitted in the open end of the housing, one end of which projects into and is in fluid communication with the interior of the housing. Thus, in one aspect, the end of the nib that is received by the interior of the elongated housing is also in fluid communication with the interior of the marking agent reservoir.

The marking agent reservoir may, in one aspect, comprise an opening for delivering suitable marking agent from the reservoir to the nib. Thus, in one aspect, the nib may be a conventional felted foam tip nib which is capable of receiving marking agent from the marking agent reservoir and shaped and sized such that when the nib is drawn across the tissue of a surgical patient, the marking agent may then be deposited onto the surface of the patient's tissue, leaving a desired mark. In another aspect, it is contemplated that the nib itself may also be the marking agent reservoir. For example, a conventional felt tip nib could be pre-loaded with a suitable marking agent such that the marking pen is operable only until the marking agent loaded into the nib is either deposited onto a desired surface or the marking agent loaded within the nib dries up after being exposed to air.

In another aspect, the marking agent reservoir may comprise only a sufficient amount of marking agent for a single use of the surgical marking pen. Further, the marking pen housing may be sized and shaped to form a friction fit within a cap member sized and shaped to cover the marking nib and to thereby prevent the marking agent from drying out and rendering the marking pen inoperable.

In still another embodiment, the housing of the marking device may further comprise a sealed frangible glass or plastic tube fitted in the interior of the housing, which exterior dimensions relative to the interior dimension of the housing is such that the tube may be cracked or broken and the liquid contents thereof released into the interior of the housing by bending or squeezing the sidewall of the housing. According to this embodiment, the nib of the marking pen will not be in fluid communication with a marking agent until the frangible marking agent reservoir is ruptured, at which time a suitable marking agent would be released and would saturate the nib with the marking agent, thereby permitting the transfer of the resulting marking agent onto the skin or surface of a surgical patient.

As previously described herein, a suitable marking agent for use on a surgical site marking pen may be any one of those marking agents approved for topical use on a patient's skin. In one aspect, it is desirable for the marking agent to be temporary (i.e. biodegradable) such that the patient is not left with a prolonged marking. To this end, suitable marking agents may include one or more inks that have been approved by the United States Food and Drug Administration (FDA), including without limitation, inks that have been approved for use with food and food packaging. In one aspect, the marking agent is an ink composition such as methylene blue, brilliant green and/or gentian violet. Furthermore, it should be understood that any desired color may be used provided that the image, when transferred to the patient's skin, provides a sufficient marking that is clearly visible to the surgeon.

As one of ordinary skill in the art will appreciate, a marking pen 60 according to the instant invention may be scaled to any desired size and shape so long as the marking pen is capable of providing a desired mark on the tissue of a surgical patient. Further, the spread of infectious disease may be a serious concern to one of ordinary skill in the art and therefore measures are frequently taken to minimize these occurrences. It may therefore be desirable for a marking pen according to the instant invention to be disposable and thus disposed of after a single use. Accordingly, in one aspect, one or more surgical marking pens may be packaged in a manner that is capable of providing an indication to a prospective user as to whether a marking pen has previously been used. For example, with references to FIGS. 5 through 8, one or more surgical marking pens 60 of the instant invention may be individually packaged in a conventional blister pack 62. To this end, a conventional a blister pack may comprise a plurality of bubbles or blisters 64 adapted to receive one or more marking pens. Further, the bubble or blister may be sealed with a conventional blister pack lid or seal 66, such as a conventional foil seal. Thus, a broken blister or seal covering a blister may serve as evidence of possible prior usage of a marking pen.

It is further contemplated that a marking pen according to the instant invention may be used to create freehand marks on a surgical patient. Such marks may include, without limitation, lines, symbols, words, shapes and any other desired marks. However, in an alternative aspect, it is also contemplated that the marking pens of the instant invention may be used in connection with a surgical site marking stencil. Thus, a kit 50 according to the present invention may further comprise one or more conventional stencil devices.

A stencil device 70 suitable for use in connection with a kit of the present invention may be any conventional stencil that is capable of use in connection with marking a surgical patient. In one aspect, and as depicted in FIG. 9, a stencil 70 according to the present invention may comprise a relatively planar stencil base frame 72 defining one or more cut-outs 74 corresponding to a desired mark or plurality of marks to be stenciled onto a surgical patient. As illustrated, the stencil of FIG. 9 depicts an exemplary stencil 70 with a capital letter “YES” cut-out in the stencil base. In use, the stencil would be placed upon a surgical patient and the skin of the patient apparent through the cut out portion would be colored with a suitable marking agent. In one aspect, the marking agent may be supplied by a surgical marking pen described above. An exemplary and commercially available stencil suitable for use in the instant invention are manufactured by and can be obtained from Sheenya Enterprises Co. Ltd, Feng Shan City, Kaohsiung, Taiwan, R.O.C.

It will be appreciated that a stencil according to the present invention may contain a cut out or plurality of cut outs sized and shaped to provide any desired marking. For example, a string of letters (e.g., ABCs, a person's name, words, numbers, shapes, etc.). In one aspect, the stencil contains a string of letters “Y E S” such that the stencil enables a user to mark the patient with the word “yes.” It should also be understood that the stencil can provide a desired word or phrase in any language. For example, and without limitation, the stencil may provide a phrase or term from, for example, the Spanish, French, German, Russian, Chinese, Japanese, and/or Arabic languages. In still another aspect, it is also envisioned that the image to be transferred may comprise a phrase or term from two or more languages.

If desired, a stencil may further contain an FDA approved adhesive material 76 to facilitate its retention of the surface of a patient during the marking process. When the stenciling task is complete, the stencil device may then be easily removed from the surface or skin of the patient and disposed of appropriately.

In still another aspect of the present invention, a kit is provided that comprises at least one conventional temporary surgical site marking tattoo or decal 80. To this end a suitable tattoo or decal for use in the present invention includes, without limitation, any conventional tattoo or decal that utilizes transfer sheets, decals, and the like. Examples of temporary tattoos as used herein, are disclosed in, for example, U.S. Pat. Nos. 4,522,864; and 6,074,721, the contents of which are hereby incorporated by reference. Other suitable exemplary tattoo devices for use in the instant invention include those manufactured by Tattoo Mfg. Inc., Tucson, Ariz. and the TAT Marker™, available from OP-Marks, Inc., Athens, Ga., USA.

A conventional tattoo may be advantageously applied to the skin of a patient using known tattoo application techniques by medical/surgical personnel prior to surgery or in connection with providing further medical treatment to such patient, e.g., as part of a surgical preparatory procedure. A conventional tattoo preferably utilizes FDA-approved materials, e.g., FDA-approved inks and substrates, to enhance safety and efficacy. It is further preferred that the ink and substrate associated with a tattoo be fabricated using hypoallergenic materials, thereby minimizing the risk of infection or other adverse effect.

The tattoo 80 may be used to transfer any desired graphic, alpha, numeric and/or alpha-numeric combination that is suitable for use in a manner that would clearly indicate the correct site of the surgical procedure. In one aspect, it is further envisioned that the desired image may be reflective of the actual surgical procedure to be performed. To this end, it should also be appreciated that any alpha, numeric, or alpha-numeric image, phrase or term may further be configured in any desired font, including without limitation, such fonts as Times Roman, Courier, Arial and the like as well as in conjunction with any one or more special effect such as bold face print, all capital letters, italics, underlined text, and the like. It should also be understood that the tattoo can provide any desired word or phrase from and in any desired language. For example, and without limitation, the tattoo can provide an image comprising a phrase or term from, for example, the Spanish, French, German, Russian, Chinese, Japanese, and/or Arabic languages. In still another aspect, it is also envisioned that the image to be transferred may comprise a phrase or term from two or more languages. With reference to FIG. 10, a tattoo 80 is depicted that includes the word “yes” positioned within a circle.

One of ordinary skill in the art will appreciate that certain circumstances, surgical site locations, and surgical procedures are more appropriate for certain marking devices, procedures and protocols than others. For example, a surgical procedure in a cosmetically visible location, such as on the face of a patient, may not lend itself to the use of a marking assembly or device that utilizes an ink composition as the ink composition may remain visible for a significant duration of time. Thus, for example, in some instances where prompt removal of a surgical site marking is desired, the use of a temporary tattoo as described above may be desired. Alternatively, in some locations, the use of a stencil and marking pen may be more particularly suited due to the size or location of a particular surgical site. Therefore, in another aspect, the methods of the present invention may further comprise a selection step, wherein, for example, a user identifies the particular surgical site to be marked and then selects one or more desired marking devices such as those described herein. Accordingly, an exemplary and non-limiting list of suggested surgical site marking protocols for a variety of procedures are set forth in Table 1 below:

TABLE 1

| | | | | Pen & | | Suggested Mark |
|---|---|---|---|---|---|---|
| Specialty | Procedure | Mark | Tattoo | Stencil | Print Member | Location |
| Neurosurgery | Laminectomy | YES | | + | + | |
| | Nerve | YES | | + | + | |
| | Decompression | | | | | |
| Ophthalmology | Cataract | YES | + | | | above eyebrow |
| | Blepharoplasty | YES | + | | | Above eyebrow (unless |
| | | | | | | marked by surgeon) |
| | Enucleation | YES | | | | |
| ENT | Myringotomy | YES | + | | | Pre-tragal |
| | Tubes | | | | | |
| Plastic Surgery | Blepharoplasty | YES | + | | | (unless marked by |
| | | | | | | surgeon) |
| | Otoplasty | YES | + | + | + | |
| C-T Surgery | Thoracotomy | YES | | + | + | Anterior chest |
| General Surgery | Breast Biopsy | YES | | + | + | |
| | Lumpectomy/ | YES | | + | + | |
| | Mastectomy | | | | | |
| | Sential Node | YES | | + | + | Anterior axillary fold |
| | Biopsy | | | | | |
| | lng Hernia Repair | YES | | + | + | Above hernia site |
| | (open) | | | | | |
| OB-GYN | Oophorectomy | YES | | + | + | Anterior abdomen on |
| | | | | | | side |
| Vascular Surgery | Carotid | YES | | + | + | On medial clavicle; |
| | Endarterectomy | | | | | avoid pressure on |
| | | | | | | carotid or volar forearm |
| | AV Graft | YES | | + | + | On Volar forearm |
| | Fem-Pop Bypass | YES | | + | + | Above knee |
| | Amputation | YES | | + | + | Above knee/mid thigh |
| Orthopedic | ORIF | YES | | + | + | Mark dorsal side of |
| Surgery | | | | | | joint, avoid open |
| | | | | | | wounds |
| | Ext. Fixation | YES | | + | + | |
| | Arthroscopy | YES | | + | + | Mark side of joint |
| | CTR | YES | | + | + | Central palm |
| | Amputation | YES | | + | + | Above knee/mid thigh |

In still another aspect, the present invention provides a method for non-permanently marking the proper place for a surgical procedure to be performed on a patient's body, the method comprising the steps of positioning an image transfer marking device impregnated with a marking agent over a predetermined location for a surgical procedure to be performed on a patient's body and contacting the image transfer marking device to the patient's body to deposit the marking agent onto the patient's body, thereby marking the location of a surgical procedure to be performed on the patient.

In one aspect, the method comprises identifying a surgical patient in need of having a surgical site marked; identifying a surgical site on the identified surgical patient; providing a print member having a top surface and a male protrusion formed thereon, the male protrusion having a distal end forming a print face sized and shaped to define a mirror image of a desired image to be transfer printed onto a surgical patient, wherein the print face is at least partially loaded with a suitable marking agent; and contacting the at least partially loaded print face with the identified surgical site to thereby deposit at least a portion of the loaded marking agent onto the surgical site and to provide the desired transfer printed image formed by the deposited marking agent.

To this end, the print member may be provided as part of a marking assembly described herein, further comprising a storage subassembly comprising i) a lid; ii) a tray, comprising a peripheral edge surface adapted to releasably receive a portion of the lid and wherein the tray defines a recess sized and shaped to releasably receive the print member in overlying registration with a recess surface formed therein; and iii) a marking agent reservoir deposited on a portion of the recess surface of the tray, the marking agent reservoir being loaded with a suitable marking agent. Once again, when the print member is received within the tray recess, the print face formed by the distal end of the male protrusion is in overlying registration with the marking agent reservoir.

It should be appreciated that the surgical site marking assembly as described and depicted in the forgoing specification and figures is not limited to use in connection with conventional two stage image transfer devices that require manual loading of the marking agent onto the raised image of the image transfer marking device. Accordingly, in another aspect of the present invention, it is envisioned that the print face of the print member may be used in connection with any image transfer device known in the art, including without limitation, a self-inking image transfer device and/or an automated image transfer device.

While this invention has been described in connection with specific aspects, features and embodiments, it is not intended to limit the scope of the invention to the particular aspects, features and embodiments set forth, but on the contrary, it is intended to cover such alternatives, modifications, and equivalents as may be included within the spirit and scope of the invention and that will become apparent to one of ordinary skill in the art upon practicing same.

1. A surgical site marking assembly, comprising;
a print member having a top surface and a male protrusion formed thereon, the male protrusion having a distal end forming a print face sized and shaped to define a mirror image of a desired image to be transfer printed onto a surgical patient in a manner indicative of a surgical site location; and
a storage subassembly, comprising: i) a lid; ii) a tray, comprising a peripheral edge surface adapted to releasably receive a portion of the lid and wherein the tray defines a recess sized and shaped to releasably receive the print member in overlying registration with a recess surface formed therein; and iii) a marking agent reservoir deposited on a portion of the recess surface of the tray, the marking agent reservoir being loaded with a suitable marking agent;
wherein, when the print member is received within the tray recess, the print face formed by the distal end of the male protrusion is in overlying registration with the marking agent reservoir.

2. The surgical site marking assembly of claim 1, wherein the print member further comprises a print member body, wherein the top surface is formed on a portion of the print member body and, wherein, when the print member is received within the tray recess, the print member is restrained by a friction fit formed between at least a portion of the print member body and at least a portion of the tray recess.

3. The surgical site marking assembly of claim 1, further comprising a guard member positioned on at least a portion of the marking agent reservoir, the guard member defining an opening sized and shaped to receive the male protrusion of the print member.

4. The surgical site marking assembly of claim 1, wherein, when the print member is received within the tray recess, the print face is in fluid communication with at least a portion of the marking agent loaded within the marking agent reservoir.

5. The surgical site marking assembly of claim 1, wherein the print face is shaped with the mirror image of the word “YES” such that the desired image to be transfer printed onto a patient's skin comprises the word “YES.”

6. The surgical site marking assembly of claim 5, wherein the print face is further shaped with a border circumferentially surrounding the mirror image of the word “YES” such that the desired image to be transfer printed onto a patient's skin comprises the word “YES” surrounded by a border.

7. The surgical site marking assembly of claim 1, wherein the suitable marking agent is approved by the United States Food and Drug Administration.

8. The surgical site marking assembly of claim 1, wherein the lid is releasably affixed to at least a portion of the peripheral edge surface of the storage subassembly by a hermetic seal.

9. The surgical site marking assembly of claim 1, wherein the marking agent reservoir comprises a foam pad.

10. A method for marking a surgical site on a surgical patient, comprising:
a) identifying a surgical patient in need of having a surgical site marked;
b) identifying a surgical site on the identified surgical patient;
c) providing a print member having a top surface and a male protrusion formed thereon, the male protrusion having a distal end forming a print face sized and shaped to define a mirror image of a desired image to be transfer printed onto a surgical patient, wherein the print face is at least partially loaded with a suitable marking agent; and
d) contacting the at least partially loaded print face with the identified surgical site to thereby deposit at least a portion of the loaded marking agent onto the surgical site and to provide the desired transfer printed image formed by the deposited marking agent.

11. The method of claim 10, wherein step c) further comprises:
providing a storage subassembly, the storage subassembly comprising i) a lid; ii) a tray, comprising a peripheral edge surface adapted to releasably receive a portion of the lid and wherein the tray defines a recess sized and shaped to releasably receive the print member in overlying registration with a recess surface formed therein; and iii) a marking agent reservoir deposited on a portion of the recess surface of the tray, the marking agent reservoir being loaded with a suitable marking agent;
wherein, when the print member is received within the tray recess, the print face formed by the distal end of the male protrusion is in overlying registration with the marking agent reservoir.

12. The method of claim 10, wherein the print face is shaped with the mirror image of the word “YES” such that the desired image to be transfer printed onto a surgical patient comprises the word “YES.”

13. The method of claim 12, wherein the print face is further shaped to provide a border circumferentially surrounding the mirror image of the word “YES” such that the desired image to be transfer printed onto a surgical patient comprises the word “YES” circumferentially surrounded by a border.

14. The method of claim 10, wherein the suitable marking agent is approved by the United States Food and Drug Administration.

15. A surgical site marking kit comprising,
a) at least one surgical site marking assembly comprising a print member having a top surface and a male protrusion formed thereon, the male protrusion having a distal end forming a print face sized and shaped to define a mirror image of a desired image to be transfer printed onto a surgical patient in a manner indicative of a surgical site location; and
b) at least one surgical site marking device selected from the group consisting of a surgical site marking pen, a surgical site marking stencil, and a temporary surgical site marking tattoo.

16. The kit of claim 15, wherein the kit comprises at least one surgical marking pen.

17. The kit of claim 16, wherein the kit comprises a plurality of surgical marking pens and wherein at least one surgical marking pen of the plurality of marking pens is packaged in a blister pack.

18. The kit of claim 16, wherein the kit comprises at least one surgical marking stencil.

19. The kit of claim 15, wherein the kit comprises at least one temporary surgical site marking tattoo.

20. The kit of claim 16, wherein the kit comprises at least one temporary surgical site marking tattoo.

21. The kit of claim 15, wherein the kit further comprises at least one marking agent reservoir loaded with a suitable marking agent, the marking agent reservoir being adapted to load the print face of the print member with the suitable marking agent.

22. The kit of claim 15, wherein the surgical site marking assembly further comprises:
a storage subassembly, comprising a lid; a tray, comprising a peripheral edge surface adapted to releasably receive a portion of the lid and wherein the tray defines a recess sized and shaped to releasably receive the print member in overlying registration with a recess surface formed therein; and a marking agent reservoir deposited on a portion of the recess surface of the tray, the marking agent reservoir being loaded with a suitable marking agent;
wherein, when the print member is received within the tray recess, the print face formed by the distal end of the male protrusion is in overlying registration with the making agent reservoir.

23. The kit of claim 15, further comprising a storage housing for housing the at least one surgical site marking assembly of a) and the at least one surgical site marking device of b).
